(19) United States
(12) Patent Application Publication
(10) Pub. No.: US 2014/0297202 A1
(43) Pub. Date: Oct. 2, 2014

(54) NONDESTRUCTIVE METHOD TO DETERMINE CRYSTALLINITY IN AMORPHOUS ALLOY

(71) Applicants: Stephen P. Zadesky, Portola Valley, CA (US); Christopher D. Prest, San Francisco, CA (US); Theodore A. Waniuk, Lake Forest, CA (US)

(72) Inventors: Stephen P. Zadesky, Portola Valley, CA (US); Christopher D. Prest, San Francisco, CA (US); Theodore A. Waniuk, Lake Forest, CA (US)

(73) Assignee: Apple Inc., Cupertino, CA (US)

(21) Appl. No.: 14/237,093

(22) PCT Filed: Aug. 5, 2011
(86) PCT No.: PCT/US2011/046715

§ 371 Date: Apr. 1, 2014

(51) Int. Cl. G01N 27/14; G01N 33/20

(52) U.S. Cl. CPC G01N 27/14; G01N 33/20
USPC 702/28; 702/27

(57) ABSTRACT

One embodiment provides a method of determining an unknown degree of crystallinity, the method comprising: constructing a master curve plot comprising a plurality of reference curves, each reference curve representing a relationship between electrical resistivity and temperature for one of a plurality of reference alloy samples having a chemical composition and various pre-determined degrees of crystallinity; for an alloy specimen having the chemical composition and the unknown degree of crystallinity, obtaining a curve representing the electrical resistivity and temperature thereof; and determining the unknown degree of crystallinity by comparing the curve to the master curve plot.

All publications, patents, and patent applications cited in this Specification are hereby incorporated by reference in their entirety.

### BACKGROUND

A large portion of the metallic alloys in use today are processed by solidification casting, at least initially. The metallic alloy is melted and cast into a metal or ceramic mold, where it solidifies. The mold is stripped away, and the cast metallic piece is ready for use or further processing. The as-cast structure of most materials produced during solidification and cooling depends upon the cooling rate. There is no general rule for the nature of the variation, but for the most part the structure changes only gradually with changes in cooling rate. On the other hand, for the bulk-solidifying amorphous alloys the change between the amorphous state produced by relatively rapid cooling and the crystalline state produced by relatively slower cooling is one of kind rather than degree—the two states have distinct properties.

Bulk-solidifying amorphous alloys, or bulk metallic glasses (“BMG”), are a recently developed class of metallic materials. These alloys may be solidified and cooled at relatively slow rates, and they retain the amorphous, non-crystalline (i.e., glassy) state at room temperature. This amorphous state can be highly advantageous for certain applications. If the cooling rate is not sufficiently high, crystals may form inside the alloy during cooling, so that the benefits of the amorphous state are lost. For example, one risk with the creation of bulk amorphous alloy parts is partial crystallization due to either slow cooling or impurities in the raw material. Thus, ensuring a high degree of amorphicity (and, conversely, a low degree of crystallinity) can be important in the quality control of a BMG fabrication process.

Currently, the methods to measure the degree of crystallinity can include bending test, x-ray radiography, and etching. However, all of these pre-existing techniques are destructive to the measurement specimens. The most common current method for screening relies on either a destructive strength test, or sectioning and subsequent visual screening for crystallization. As a result, for a BMG part (e.g., a casing) that needs to be measured for its degree of crystallinity, it needs to first be significantly altered (e.g., sectioned, published, and/or ground to a powder form).

Thus, a need exists to develop methods that can provide a measurement of the degree of crystallinity of a BMG non-destructively, whereby facilitating quality control of its fabrication process.

### SUMMARY

One embodiment provides a method of determining an unknown degree of crystallinity, the method comprising: constructing a master curve plot comprising a plurality of reference curves, each reference curve representing a relationship between electrical resistivity and temperature for one of a plurality of reference alloy samples having a chemical composition and various predetermined degrees of crystallinity; for an alloy specimen having the chemical composition and the unknown degree of crystallinity, obtaining a curve representing the electrical resistivity and temperature thereof; and determining the unknown degree of crystallinity by comparing the curve to the master curve plot.

Another embodiment provides a method of determining an unknown degree of crystallinity, the method comprising: providing a plurality of reference alloy samples of a chemical composition, each of the plurality having a predetermined degree of crystallinity; for each of the plurality, measuring a relationship between electrical resistivity and temperature to obtain a reference curve representing the relationship; constructing a master curve plot comprising at least some of the reference curves; providing an alloy specimen having the chemical composition and the unknown degree of crystallinity; for the specimen, measuring a relationship between the electrical resistivity and temperature to obtain a curve representing the relationship; and determining the unknown degree of crystallinity for the specimen by comparing the curve to the master curve plot.

An alternative embodiment provides a method of determining an unknown degree of crystallinity, the method comprising: providing a master curve plot comprising a plurality of reference curves, each reference curve representing a respective relationship between electrical resistivity and temperature of one of a plurality of reference samples, having a chemical composition and various predetermined degrees of crystallinity; measuring an electrical resistivity of an alloy specimen at a first location thereof, wherein the alloy specimen has the chemical composition and the unknown degree of crystallinity; and determining the unknown degree of crystallinity by comparing the resistivity to the master curve plot.

Another embodiment provides a method of evaluating an alloy specimen, comprising: measuring the electrical resistivity of the alloy specimen at at least two temperatures; determining a slope of a line connecting the electrical resistivity measurements on a resistivity versus temperature plot; and evaluating the alloy specimen based on the slope.

### BRIEF DESCRIPTION OF THE DRAWINGS

FIG. 1 provides an illustrative plot showing the relationship between electrical resistivity and temperature for three illustrative amorphous alloys, having respective degrees of crystallinity, X1, X2, X3, X4, X5, with X1<X2<X3<X4<X5 in one embodiment.

FIG. 2 provides an illustrative plot showing the relationship between electrical resistivity and temperature for three illustrative amorphous alloys, having respective degrees of crystallinity, X1, X2, and X3, with X1<X2<X3 in another embodiment. Each of the curves is normalized by the data obtained from an alloy of the same composition with 100% amorphicity.

FIGS. 3(a)-3(b) illustrate the different locations at which the presently described measurement and determination methods can be applied. FIG. 3(a) shows that the determination can be applied to different locations along one single axis, whereas FIG. 3(b) shows that the determination can be applied to different locations on a cross-sectional plane.

FIG. 4 provides a flow diagram showing the steps of non-destructively determining the crystallinity of an alloy specimen in one embodiment.

FIG. 5 shows a schematic diagram showing the use of the electrical resistivity vs. temperature relationship (not to scale) as a quality control mechanism in one embodiment.

FIG. 6(a)-6(c) show a schematic diagram of the relationship between electrical resistivity and temperature (not to scale) as a result of two measurements for three different samples in one embodiment.

### DETAILED DESCRIPTION

### Phase

The term “phase” herein can refer to one that can be found in a thermodynamic phase diagram. A phase is a region of space (e.g., a thermodynamic system) throughout which all physical properties of a material are essentially uniform. Examples of physical properties include density, index of refraction, chemical composition and lattice periodicity. A simple description is that a phase is a region of material that is chemically uniform, physically distinct, and/or mechanically separable. For example, in a system consisting of ice and water in a glass jar, the ice cubes are one phase, the water is a second phase, and the humid air over the water is a third phase. The glass of the jar is another separate phase. A phase can refer to a solid solution, which can be a binary, tertiary, quaternary, or more, solution, or a compound, such as an intermetallic compound. As another example, an amorphous phase is distinct from a crystalline phase.

### Metal, Transition Metal, and Non-Metal

The term “metal” refers to an electropositive chemical element. The term “element” in this Specification refers generally to an element that can be found in a Periodic Table. Physically, a metal atom in the ground state contains a partially filled band with an empty state close to an occupied state. The term “transition metal” is any of the metallic elements within Groups 3 to 12 in the Periodic Table that have an incomplete inner electron shell and serve as transitional links between the most and the least electropositive in a series of elements. Transition metals are characterized by multiple valences, colored compounds, and the ability to form stable complex ions. The term “nonmetal” refers to a chemical element that does not have the capacity to lose electrons and form a positive ion.

Depending on the application, any suitable nonmetal elements, or their combinations, can be used. The alloy composition can comprise multiple nonmetal elements, such as at least two, at least three, at least four, or more, nonmetal elements. A nonmetal element can be any element that is found in Groups 13-17 in the Periodic Table. For example, a nonmetal element can be any one of F, Cl, Br, I, At, O, S, Se, Te, Po, N, P, As, Sb, Bi, C, Si, Ge, Sn, Pb, and B. Occasionally, a nonmetal element can also refer to certain metalloids (e.g., B, Si, Ge, As, Sb, Te, and Po) in Groups 13-17. In one embodiment, the nonmetal elements can include B, Si, C, P, or combinations thereof. Accordingly, for example, the alloy composition can comprise a boride, a carbide, or both.

A transition metal element can be any of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubniuni, seaborgium, bohrium, hassium, meitnerium, ununnilium, unununium, and ununbium. In one embodiment, a BMG containing a transition metal element can have at least one of Sc, Y, La, Ac, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, and Hg. Depending on the application, any suitable transitional metal elements, or their combinations, can be used. The alloy composition can comprise multiple transitional metal elements, such as at least two, at least three, at least four, or more, transitional metal elements.

The presently described alloy or alloy “sample” or “specimen” alloy can have any shape or size. For example, the alloy can have a shape of a particulate, which can have a shape such as spherical, ellipsoid, wire-like, rod-like, sheet-like, flake-like, or an irregular shape. The particulate can have any suitable size. For example, it can have an average diameter of between about 1 micron and about 100 microns, such as between about 5 microns and about 80 microns, such as between about 10 microns and about 60 microns, such as between about 0.15 microns and about 50 microns, such as between about 15 microns and about 45 microns, such as between about 20 microns and about 40 microns, such as between about 25 microns and about 35 microns. For example, in one embodiment, the average diameter of the particulate is between about 25 microns and about 44 microns. In some embodiments, smaller particulates, such as those in the nanometer range, or larger particulates, such as those bigger than 100 microns, can be used.

The alloy sample or specimen can also be of a much larger dimension. For example, it can be a bulk structural component, such as an ingot, housing/casing of an electronic device or even a portion of a structural component that has dimensions in the millimeter, centimeter, or meter range.

### Solid Solution

The term “solid solution” refers to a solid form of a solution. The term “solution” refers to a mixture of two or more substances, which may be solids, liquids, gases, or a combination of these. The mixture can be homogeneous or heterogeneous. The term “mixture” is a composition of two or more substances that are combined with each other and generally capable of being separated. Generally, the two or more substances are not chemically combined with each other.

### Alloy

In some embodiments, the alloy powder composition described herein can be fully alloyed. In one embodiment, an “alloy” refers to a homogeneous mixture or solid solution of two or more metals, the atoms of one replacing or occupying interstitial positions between the atoms of the other; for example, brass is an alloy of zinc and copper. An alloy, in contrast to a composite, can refer to a partial or complete solid solution of one or more elements in a metal matrix, such as one or more compounds in a metallic matrix. The term “alloy” herein can refer to both a complete solid solution alloy that can give single solid phase microstructure and a partial solution that can give two or more phases.

Thus, a fully alloyed alloy can have a homogenous distribution of the constituents, be it a solid solution phase, a compound phase, or both. The term “fully alloyed” used herein can account for minor variations within the error tolerance. For example, it can refer to at least 90% alloyed, such as at least 95% alloyed, such as at least 99% alloyed, such as at least 99.5% alloyed, such as at least 99.9% alloyed. The percentage herein can refer to either volume percent or weight percentage, depending on the context. These percentages can be balanced by impurities, which can be in terms of composition or phases that are not a part of the alloy.

### Amorphous or Non-Crystalline Solid

An “amorphous” or “non-crystalline solid” is a solid that lacks lattice periodicity, which is characteristic of a crystal. As used herein, an “amorphous solid” includes “glass” which is an amorphous solid that softens and transforms into a liquid-like state upon heating through the glass transition phase. Generally, amorphous materials lack the long-range order characteristic of a crystal, though they can possess some short-range order at the atomic length scale due to the nature of chemical bonding. The distinction between amorphous solids and crystalline solids can be made based on lattice periodicity as determined by structural characterization techniques such as x-ray diffraction and transmission electron microscopy.

The terms “order” and “disorder” designate the presence or absence of some symmetry or correlation in a many-particle system. The terms “long-range order” and “short-range order” distinguish order in materials based on length scales.

The strictest form of order in a solid is lattice periodicity: a certain pattern (the arrangement of atoms in a unit cell) is repeated again and again to form a translationally invariant tiling of space. This is the defining property of a crystal. Possible symmetries have been classified in 14 Bravais lattices and 230 space groups.

Lattice periodicity implies long-range order. If only one unit cell is known, then by virtue of the translational symmetry it is possible to accurately predict all atomic positions at arbitrary distances. The converse is generally true, except, for example, in quasi-crystals that have perfectly deterministic tilings but do not possess lattice periodicity.

Long-range order characterizes physical systems in which remote portions of the same sample exhibit correlated behavior. This can be expressed as a correlation function, namely the spin-spin correlation function: G(x,x′)=<s(x), s(x′)>.

In the above function, s is the spin quantum number and x is the distance function within the particular system. This function is equal to unity when x=x′ and decreases as the distance |x−x′| increases. Typically, it decays exponentially to zero at large distances, and the system is considered to be disordered. If, however, the correlation function decays to a constant value at large |x−x′|, then the system can be said to possess long-range order. If it decays to zero as a power of the distance, then it can be called quasi-long-range order. Note that what constitutes a large value of |x−x′| is relative.

A system can be said to present quenched disorder when some parameters defining its behavior are random variables that do not evolve with time (i.e., they are quenched or frozen)—e.g., spin glasses. It is opposite to annealed disorder, where the random variables are allowed to evolve themselves. Embodiments herein include systems comprising quenched disorder.

The alloy described herein can be crystalline, partially crystalline, amorphous, or substantially amorphous. For example, the alloy sample/specimen can include at least some crystallinity, with grains/crystals having sizes in the nanometer and/or micrometer ranges. Alternatively, the alloy can be substantially amorphous, such as fully amorphous. In one embodiment, the alloy powder composition is at least substantially not amorphous, such as being substantially crystalline, such as being entirely crystalline.

In one embodiment, the presence of a crystal or a plurality of crystals in an otherwise amorphous alloy can be construed as a “crystalline phase” therein. The degree of crystallinity (or “crystallinity” for short in some embodiments) of an alloy can refer to the amount of the crystalline phase present in the alloy. The degree can refer to, for example, a fraction of crystals present in the alloy. The fraction can refer to volume fraction or weight fraction, depending on the context. A measure of how “amorphous” an amorphous alloy is can be amorphicity. Amorphicity can be measured in terms of a degree of crystallinity. For example, in one embodiment, an alloy having a low degree of crystallinity can be said to have a high degree of amorphicity. In one embodiment, for example, an alloy having 60 vol % crystalline phase can have a 40 vol % amorphous phase.

### Amorphous Alloy or Amorphous Metal

An “amorphous alloy” is an alloy having an amorphous content of more than 50% by volume, preferably more than 90% by volume of amorphous content, more preferably more than 95% by volume of amorphous content, and most preferably more than 99% to almost 100% by volume of amorphous content. Note that, as described above, an alloy high in amorphicity is equivalently low in degree of crystallinity. An “amorphous metal” is an amorphous metal material with a disordered atomic-scale structure. In contrast to most metals, which are crystalline and therefore have a highly ordered arrangement of atoms, amorphous alloys are non-crystalline. Materials in which such a disordered structure is produced directly from the liquid state during cooling are sometimes referred to as “glasses.” Accordingly, amorphous metals are commonly referred to as “metallic glasses” or “glassy metals.” In one embodiment, a “bulk metallic glass” (“BMG”) can refer to an alloy, of which the microstructure is at least partially amorphous. However, there are several ways besides extremely rapid cooling to produce amorphous metals, including physical vapor deposition, solid-state reaction, ion irradiation, melt spinning, and mechanical alloying. Amorphous alloys can be a single class of materials, regardless of how they are prepared.

Amorphous metals can be produced through a variety of quick-cooling methods. For instance, amorphous metals can be produced by sputtering molten metal onto a spinning metal disk. The rapid cooling, on the order of millions of degrees a second, can be too fast for crystals to form, and the material is thus “locked in” a glassy state. Also, amorphous metals/alloys can be produced with critical cooling rates low enough to allow formation of amorphous structure in thick layers—e.g., bulk metallic glasses.

The terms “bulk metallic glass” (“BMG”), bulk amorphous alloys, and bulk solidifying amorphous alloys are used interchangeably herein. They refer to amorphous alloys having the smallest dimension at least in the millimeter range. For example, the dimension can be at least about 0.5 mm, such as at least about 1 mm, such as at least about 2 mm, such as at least about 4 mm, such as at least about 5 mm, such as at least about 6 mm, such as at least about 8 mm, such as at least about 10 mm, such as at least about 12 mm. Depending on the geometry, the dimension can refer to the diameter, radius, thickness, width, length, etc. A BMG can also be a metallic glass having at least one dimension in the centimeter range, such as at least about 1.0 cm, such as at least about 2.0 cm, such as at least about 5.0 cm, such as at least about 10.0 cm. In some embodiments, a BMG can have at least one dimension at least in the meter range. A BMG can take any of the shapes or forms described above, as related to a metallic glass. Accordingly, a BMG described herein in some embodiments can be different from a thin film made by a conventional deposition technique in one important aspect—the former can be of a much larger dimension than the latter.

Amorphous metals can be an alloy rather than a pure metal. The alloys may contain atoms of significantly different sizes, leading to low free volume (and therefore having viscosity up to orders of magnitude higher than other metals and alloys) in a molten state. The viscosity prevents the atoms from moving enough to form an ordered lattice. The material structure may result in low shrinkage during cooling and resistance to plastic deformation. The absence of grain boundaries, the weak spots of crystalline materials in some cases, may, for example, lead to better resistance to wear and corrosion. In one embodiment, amorphous metals, while technically glasses, may also be much tougher and less brittle than oxide glasses and ceramics.

Thermal conductivity of amorphous materials may be lower than that of their crystalline counterparts. To achieve formation of an amorphous structure even during slower cooling, the alloy may be made of three or more components, leading to complex crystal units with higher potential energy and lower probability of formation. The formation of amorphous alloy can depend on several factors: the composition of the components of the alloy; the atomic radius of the components (preferably with a significant difference of over 12% to achieve high packing density and low free volume); and the negative heat of mixing the combination of components, inhibiting crystal nucleation and prolonging the time the molten metal stays in a supercooled state. However, as the formation of an amorphous alloy is based on many different variables, it can be difficult to make a prior determination of whether an alloy composition would form an amorphous alloy.

Amorphous alloys, for example, of boron, silicon, phosphorus, and other glass formers with magnetic metals (iron, cobalt, nickel) may be magnetic, with low coercivity and high electrical resistance. The high resistance leads to low losses by eddy currents when subjected to alternating magnetic fields, a property useful, for example, as transformer magnetic cores.

Amorphous alloys may have a variety of potentially useful properties. In particular, they tend to be stronger than crystalline alloys of similar chemical composition, and they can sustain larger reversible (“elastic”) deformations than crystalline alloys. Amorphous metals derive their strength directly from their non-crystalline structure, which can have none of the defects (such as dislocations) that limit the strength of crystalline alloys. For example, one modern amorphous metal, known as Vitreloy™, has a tensile strength that is almost twice that of high-grade titanium. In some embodiments, metallic glasses at room temperature are not ductile and tend to fail suddenly when loaded in tension, which limits the material applicability in reliability-critical applications, as the impending failure is not evident. Therefore, to overcome this challenge, metal matrix composite materials having a metallic glass matrix containing dendritic particles or fibers of a ductile crystalline metal can be used. Alternatively, a BMG low in element(s) that tend to cause embitterment (e.g., Ni) can be used. For example, a Ni-free BMG can be used to improve the ductility of the BMG.

Another useful property of bulk amorphous alloys is that they can be true glasses; in other words, they can soften and flow upon heating. This allows for easy processing, such as by injection molding, in much the same way as polymers. As a result, amorphous alloys can be used for making sports equipment, medical devices, electronic components and equipment, and thin films. Thin films of amorphous metals can be deposited as protective coatings via a high velocity oxygen fuel technique.

A material can have an amorphous phase, a crystalline phase, or both. The amorphous and crystalline phases can have the same chemical composition and differ only in the microstructure—i.e., one amorphous and the other crystalline. Microstructure in one embodiment refers to the structure of a material as revealed by a microscope at 25× magnification or higher. Alternatively, the two phases can have different chemical compositions and microstructures. For example, a composition can be partially amorphous, substantially amorphous, or completely amorphous.

As described above, the degree of amorphicity (and conversely the degree of crystallinity) can be measured by the fraction of crystals present in the alloy. The degree can refer to volume fraction of weight fraction of the crystalline phase present in the alloy. A partially amorphous composition can refer to a composition of at least about 5 vol % of which is of an amorphous phase, such as at least about 10 vol %, such as at least about 20 vol %, such as at least about 40 vol %, such as at least about 60 vol %, such as at least about 80 vol %, such as at least about 90 vol %. The terms “substantially” and “about” have been defined elsewhere in this application. Accordingly, a composition that is at least substantially amorphous can refer to one of which at least about 90 vol %′ is amorphous, such as at least about 95 vol %, such as at least about 98 vol %, such as at least about 99 vol %, such as at least about 99.5 vol %, such as at least about 99.8 vol %, such as at least about 99.9 vol %. In one embodiment, a substantially amorphous composition can have some incidental, insignificant amount of crystalline phase present therein.

In one embodiment, an amorphous alloy composition can be homogeneous with respect to the amorphous phase. A substance that is uniform in composition is homogeneous. This is in contrast to a substance that is heterogeneous. The term “composition” refers to the chemical composition and/or microstructure in the substance. A substance is homogeneous when a volume of the substance is divided in half and both halves have substantially the same composition. For example, a particulate suspension is homogeneous when a volume of the particulate suspension is divided in half and both halves have substantially the same volume of particles. However, it might be possible to see the individual particles under a microscope. Another example of a homogeneous substance is air where different ingredients therein are equally suspended, though the particles, gases and liquids in air can be analyzed separately or separated from air.

A composition that is homogeneous with respect to an amorphous alloy can refer to one having an amorphous phase substantially uniformly distributed throughout its microstructure. In other words, the composition macroscopically comprises a substantially uniformly distributed amorphous alloy throughout the composition. In an alternative embodiment, the composition can be of a composite, having an amorphous phase having therein a non-amorphous phase. The non-amorphous phase can be a crystal or a plurality of crystals. The crystals can be in the form of particulates of any shape, such as spherical, ellipsoid, wire-like, rod-like, sheet-like, flake-like, or an irregular shape. In one embodiment, it can have a dendritic form. For example, an at least partially amorphous composite composition can have a crystalline phase in the shape of dendrites dispersed in an amorphous phase matrix; the dispersion can be uniform or non-uniform, and the amorphous phase and the crystalline phase can have the same or a different chemical composition. In one embodiment, they have substantially the same chemical composition. In another embodiment, the crystalline phase can be more ductile than the BMG phase.

The methods described herein can be applicable to any type of amorphous alloys. Similarly, the amorphous alloys described herein as a constituent of a composition or article can be of any type. The amorphous alloy can comprise the element Zr, Hf, Ti, Cu, Ni, Pt, Pd, Fe, Mg, Au, La, Ag, Al, Mo, Nb, Be, or combinations thereof. Namely, the alloy can include any combination of these elements in its chemical formula or chemical composition. The elements can be present at different weight or volume percentages. For example, an iron “based” alloy can refer to an alloy having a non-insignificant weight percentage of iron present therein, the weight percent can be, for example, at least about 20 wt %, such as at least about 40 wt %, such as at least about 50 wt %, such as at least about 60 wt %, such as at least about 80 wt %. Alternatively, in one embodiment, the above-described percentages can be volume percentages, instead of weight percentages. Accordingly, an amorphous alloy can be zirconium-based, titanium-based, platinum-based, palladium-based, gold-based, silver-based, copper-based, iron-based, nickel-based, aluminum-based, molybdenum-based, and the like. In some embodiments, the alloy, or the composition including the alloy, can be substantially free of nickel, aluminum, or beryllium, or combinations thereof. In one embodiment, the alloy or the composite is completely free of nickel, aluminum, or beryllium, or combinations thereof.

For example, the amorphous alloy can have the formula (Zr, Ti)ₐ(Ni, Cu, Fe)_{b}(Be, Al, Si, B)_{c}, wherein a, b, and c each represents a weight or atomic percentage. In one embodiment, a is in the range of from 30 to 75, b is in the range of from 5 to 60, and c is in the range of from 0 to 50 in atomic percentages. Alternatively, the amorphous alloy can have the formula (Zr, Ti)ₐ(Ni, Cu)_{b}(Be)_{c}, wherein a, b, and c each represents a weight or atomic percentage. In one embodiment, a is in the range of from 40 to 75, b is in the range of from 5 to 50, and c is in the range of from 5 to 50 in atomic percentages. The alloy can also have the formula (Zr, Ti)ₐ(Ni, Cu)_{b}(Be)_{c}, wherein a, b, and c each represents a weight or atomic percentage. In one embodiment, a is in the range of from 45 to 65, b is in the range of from 7.5 to 35, and c is in the range of from 10 to 37.5 in atomic percentages. Alternatively, the alloy can have the formula (Zr)ₐ(Nb, Ti)_{b}(Ni, Cu)_{c}(A1)_{d}, wherein a, b, c, and d each represents a weight or atomic percentage. In one embodiment, a is in the range of from 45 to 65, b is in the range of from 0 to 10, c is in the range of from 20 to 40 and d is in the range of from 7.5 to 15 in atomic percentages. One exemplary embodiment of the aforedescribed alloy system is a Zr—Ti—Ni—Cu—Be based amorphous alloy under the trade name Vitreloy™, such as Vitreloy-1 and Vitreloy-101, as fabricated by Liquidmetal Technologies, CA, USA. Some examples of amorphous alloys of the different systems are provided in Table 1.

The amorphous alloys can also be ferrous alloys, such as (Fe, Ni, Co) based alloys. Examples of such compositions are disclosed in U.S. Pat. Nos. 6,325,868; 5,288,344; 5,368,659; 5,618,359; and U.S. Pat. No. 5,735,975, Inoue et al., Appl. Phys. Lett., Volume 71, p 464 (1997), Shen et al., Mater. Trans., JIM, Volume 42, p 2136 (2001), and Japanese Patent Application No. 200126277 (Pub. No. 2001303218 A). One exemplary composition is Fe₇₂Al₅Ga₂P₁₁C₆B₄. Another example is Fe₇₂Al₇Zr₁₀MO₅W₂B₁₅. Another iron-based alloy system that can be used in the coating herein is disclosed in U.S. Patent Application Publication No. 2010/0084052, wherein the amorphous metal contains, for example, manganese (1 to 3 atomic %), yttrium (0.1 to 10 atomic %), and silicon (0.3 to 3.1 atomic %) in the range of composition given in parentheses; and that contains the following elements in the specified range of composition given in parentheses: chromium (15 to 20 atomic %), molybdenum (2 to 15 atomic %), tungsten (1 to 3 atomic %), boron (5 to 16 atomic %), carbon (3 to 16 atomic %), and the balance iron.

The aforedescribed amorphous alloy systems can further include additional elements, such as additional transition metal elements, including Nb, Cr, V, and Co. The additional elements can be present at less than or equal to about 30 wt %, such as less than or equal to about 20 wt %, such as less than or equal to about 10 wt %, such as less than or equal to about 5 wt %. In one embodiment, the additional, optional element is at least one of cobalt, manganese, zirconium, tantalum, niobium, tungsten, yttrium, titanium, vanadium and hafnium to form carbides and further improve wear and corrosion resistance. Further optional elements may include phosphorous, germanium and arsenic, totaling up to about 2%, and preferably less than 1%, to reduce melting point. Otherwise incidental impurities should be less than about 2% and preferably 0.5%.

In some embodiments a composition having an amorphous alloy can include a small amount of impurities. The impurity elements can be intentionally added to modify the properties of the composition, such as improving the mechanical properties (e.g., hardness, strength, fracture mechanism, etc.) and/or improving the corrosion resistance. Alternatively, the impurities can be present as inevitable, incidental impurities, such as those obtained as a byproduct of processing and manufacturing. The impurities can be less than or equal to about 10 wt %, such as about 5 wt %, such as about 2 wt %, such as about 1 wt %, such as about 0.5 wt %, such as about 0.1 wt %. In some embodiments, these percentages can be volume percentages instead of weight percentages. In one embodiment, the alloy sample/composition consists essentially of the amorphous alloy (with only a small incidental amount of impurities). In another embodiment, the composition consists of the amorphous alloy (with no observable trace of impurities).

TABLE 1
Exemplary amorphous alloy compositions

| Alloy | Atm % | Atm % | Atm % | Atm % | Atm % | Atm % |
|---|---|---|---|---|---|---|
| 1 | Zr | Ti | Cu | Ni | Be | |
| | 41.20% | 13.80% | 12.50% | 10.00% | 22.50% | |
| 2 | Zr | Ti | Cu | Ni | Be | |
| | 44.00% | 11.00% | 10.00% | 10.00% | 25.00% | |
| 3 | Zr | Ti | Cu | Ni | Nb | Be |
| | 56.25% | 11.25% | 6.88% | 5.63% | 7.50% | 12.50% |
| 4 | Zr | Ti | Cu | Ni | Al | Be |
| | 64.75% | 5.60% | 14.90% | 11.15% | 2.60% | 1.00% |
| 5 | Zr | Ti | Cu | Ni | Al | |
| | 52.50% | 5.00% | 17.90% | 14.60% | 10.00% | |
| 6 | Zr | Nb | Cu | Ni | Al | |
| | 57.00% | 5.00% | 15.40% | 12.60% | 10.00% | |
| 7 | Zr | Cu | Ni | Al | Sn | |
| | 50.75% | 36.23% | 4.03% | 9.00% | 0.50% | |
| 8 | Zr | Ti | Cu | Ni | Be | |
| | 46.75% | 8.25% | 7.50% | 10.00% | 27.50% | |
| 9 | Zr | Ti | Ni | Be | | |
| | 21.67% | 43.33% | 7.50% | 27.50% | | |
| 10 | Zr | Ti | Cu | Be | | |
| | 35.00% | 30.00% | 7.50% | 27.50% | | |
| 11 | Zr | Ti | Co | Be | | |
| | 35.00% | 30.00% | 6.00% | 29.00% | | |
| 12 | Au | Ag | Pd | Cu | Si | |
| | 49.00% | 5.50% | 2.30% | 26.90% | 16.30% | |
| 13 | Au | Ag | Pd | Cu | Si | |
| | 50.90% | 3.00% | 2.30% | 27.80% | 16.00% | |
| 14 | Pt | Cu | Ni | P | | |
| | 57.50% | 14.70% | 5.30% | 22.50% | | |
| 15 | Zr | Ti | Nb | Cu | Be | |
| | 36.60% | 31.40% | 7.00% | 5.90% | 19.10% | |
| 16 | Zr | Ti | Nb | Cu | Be | |
| | 38.30% | 32.90% | 7.30% | 6.20% | 15.30% | |
| 17 | Zr | Ti | Nb | Cu | Be | |
| | 39.60% | 33.90% | 7.60% | 6.40% | 12.50% | |
| 18 | Cu | Ti | Zr | Ni | | |
| | 47.00% | 34.00% | 11.00% | 8.00% | | |
| 19 | Zr | Co | Al | | | |
| | 55.00% | 25.00% | 20.00% | | | |

### Electrical Resistivity

Electrical resistivity (p) (also known as resistivity, specific electrical resistance, or volume resistivity) is a measure of how strongly a material opposes the flow of electric current. A low resistivity indicates a material that readily allows the movement of electrical charge. The SI unit of electrical resistivity is ohm-meter (Ωm). Resistivity is an intrinsic property of a material and thus is geometrically independent. Electrical resistance (R), on the other hand, depends on the geometry of the material. For example, in one embodiment, electrical resistance is inversely proportional to the cross-sectional area of a wire but is directly proportional to the length of the wire. In other words, a short wire would have a smaller electrical resistance than a lower wire, whereas a wire with a small cross-sectional area would have a larger electrical resistance than one with a large cross-sectional area.

In general, electrical resistivity of crystalline metals and their alloys increases with temperature, while the resistivity of semiconductors decreases with increasing temperature. In both cases, electron—phonon interactions can play an important role. At high temperatures, the resistance of a metal increases linearly with temperature. As the temperature of a metal is reduced, the temperature dependence of resistivity follows a power law function of temperature. Mathematically, the temperature dependence of the resistivity ρ of a metal is given by the Bloch-Gruneisen formula:

$$ρ ( T ) = ρ ( 0 ) + {A ( \frac{T}{{⊖}_{R}} )}^{n} {\int }_{0}^{\frac{{⊖}_{R}}{T}} \frac{{x}^{n}}{( {e}^{x} - 1 ) ( 1 - {e}^{- x} )} d x$$

where ρ(0) is the residual resistivity due to defect scattering, A is a constant that depends on the velocity of electrons at the Fermi surface, the Debye radius and the number density of electrons in the metal. Θ_{R} is the Debye temperature as obtained from resistivity measurements and matches very closely with the values of Debye temperature obtained from specific heat measurements. n is an integer that depends upon the nature of interaction: n=5 implies that the resistance is due to scattering of electrons by phonons (as it is for simple metals); n=3 implies that the resistance is due to s-d electron scattering (as is the case for transition metals); n=2 implies that the resistance is due to electron—electron interaction.

As the temperature of the metal is sufficiently reduced (so as to “freeze” all the phonons), the electrical resistivity usually reaches a constant value, known as the “residual resistivity.” This value depends not only on the type of metal, but on its purity and thermal history. The value of the residual resistivity of a metal can be decided by its impurity concentration. Some materials lose all electrical resistivity at sufficiently low temperatures, due to an effect known as superconductivity.

In contrast to crystalline metals and metal alloys, metallic glasses behave quite differently with respect to the electrical resistivity. For example, in crystalline metal alloys the electrical resistance increases linearly with rising temperature, whereas in amorphous alloys, the electrical resistance does not significantly change, or even show a decrease, with increasing temperature until crystallization takes place. It is commonly known that a metallic glass exhibits an electrical resistivity that remains fairly constant or decreases with increasing temperature (i.e., a small and often negative temperature coefficient), whereas its crystalline counterpart has a large and positive temperature coefficient or resistivity. Further, the electrical resistivity (ρ) of metallic glasses can be larger than that of a crystalline alloy. For example, the p of metallic glasses can range from about 100 to about 250 μΩ·cm and can vary smoothly and continuously as the glass transition at Tg is traversed. By contrast, crystalline metal alloys can have a much smaller resistivity (e.g., 1-50 μΩ·cm) and a larger and more positive temperature coefficient.

For example, for a Zr-based alloy, the crystallization temperature (Tx) can be at around 750 K. Thus, in a ρ vs. T plot of such an alloy, the curve would appear relatively flat horizontally until 750 K, at which crystallization occurs. Thus, by measuring the electrical resistivity of a bulk metallic glass sample over a given temperature range, it may be possible to detect nondestructively the degree of crystallization/crystallinity of the sample.

### Master Curve Plot

The presently described methods to determine the degree of crystallinity can involve first constructing a master curve plot and thereafter using the plot to determine the degree of crystallinity of a specimen. The term “master curve plot” herein is used to describe a plot containing a plurality of reference curves representing a certain material property of predetermined reference samples. The property can be represented by, for example, a relationship and/or a mathematical function, as shown on a plot and/or by a mathematical expression. For example, the plot can illustrate the temperature dependence (x-axis) of electrical resistivity (and, inversely, electrical conductivity), specific density, volume, mass, thermal resistivity (and, inversely, thermal conductivity), crystallinity, etc. (on the y-axis). The parameter on the x-axis needs not to be temperature; it can be crystallinity, time, density, volume, composition, etc. Any suitable axis can be used for illustrating the material property.

As in the case of material property comparisons, a master curve plot is preferably constructed while holding a parameter of the sample materials to be compared constant, such that at least one other parameter of the sample can be compared. For example, in one embodiment, the master curve plot is alloy composition specific. Namely, the plot can be constructed for a plurality of reference alloy samples of the same composition. The reference alloy samples can have various degrees of crystallinity. In one embodiment, the master curve plot can comprise a plurality of reference curves, each reference curve representing a relationship between electrical resistivity and temperature of one of a plurality of reference samples. The reference samples can have the same chemical composition but various predetermined degrees of crystallinity.

The master curve plot (or “master plot” for short in some embodiments) can be constructed by various techniques, particularly depending on the parameters to be examined using the plot. For example, one embodiment described herein provides a master plot showing the relationship between electrical resistivity as a function of temperature for a plurality of reference samples—i.e., temperature dependence of electrical resistivity. These reference samples generally should have at least one common parameter so that they can be compared with respect to another parameter.

In one embodiment, a master curve plot is constructed for a plurality of reference alloy samples having a predetermined, known chemical composition. However, while these reference samples have the same chemical composition, they have different degrees of crystallinity. For example, some of these reference samples can be 100 vol % crystalline, 25 vol % crystalline, 50 vol % crystalline, 75 vol % crystalline, or 0 vol % crystalline (amorphous). In this embodiment, the relationship between the electrical resistivity and temperature of each of the reference samples can be measured. Such a relationship for each reference sample can be represented by a “reference” curve. Depending on the application, the data acquisition frequency can be of any value. Also, in the case where several reference samples of the same composition and degree of crystallinity are measured, the data point of these same reference samples can be represented as an average, with standard deviation (if appropriate). The data point need not be taken from various reference samples of the same composition and degree of crystallinity. For example, it can be taken from different locations of the reference sample—such a measurement can provide an indicator of the uniformity of the material property along a specific axis. Further statistical analysis can be performed based on the obtained data, if desired.

A master curve plot can be constructed by superimposing a plurality of reference curves, all having the same chemical composition but each having its own pre-determined degree of crystallinity. As described before, at a temperature below the onset of crystallization, the electrical resistivity of an alloy that is at least partially amorphous is generally independent of temperature. Namely, on a plot of electrical resistivity vs. temperature, the curve can be fairly flat until the crystallization temperature. However, as aforedescribed, in some embodiments, the electrical resistivity of an amorphous alloy can decrease with increasing degree of crystallinity.

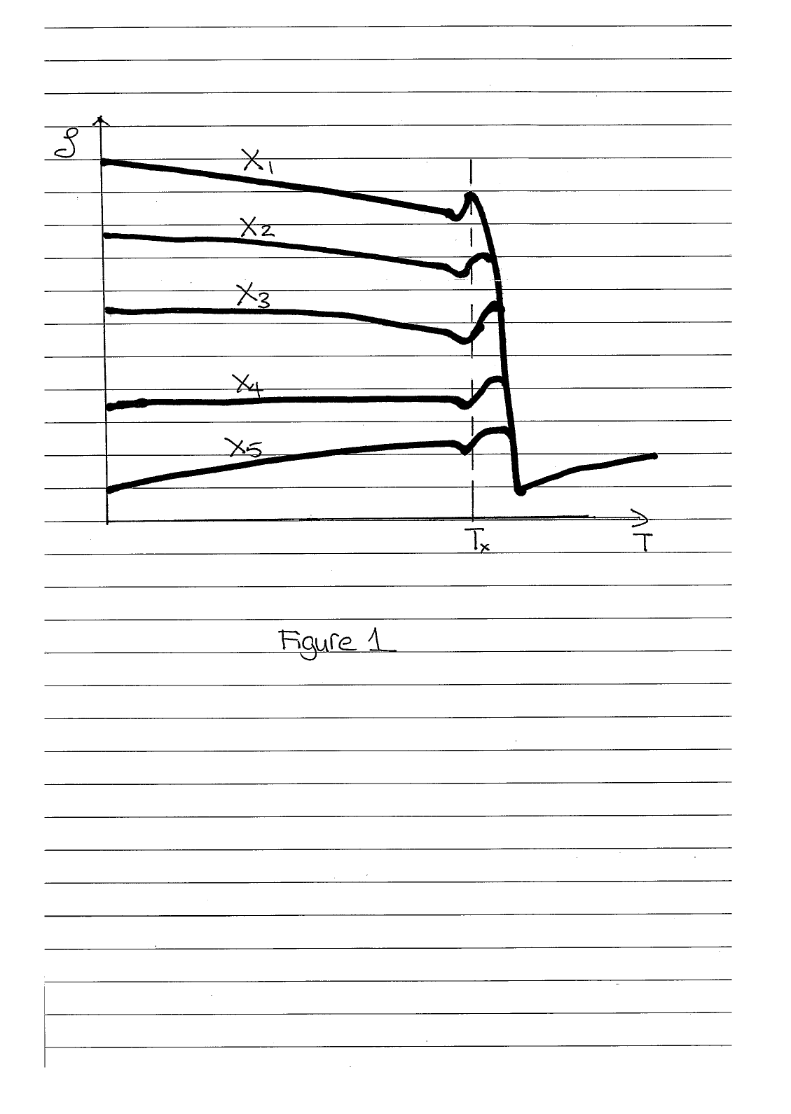

FIG. 1 provides an illustrative plot showing the electrical resistivity of three illustrative amorphous alloys, A1, A2, A3, A4, and A5 with respective degree of crystallinity X1, X2, X3, X4, and X5 in one embodiment. Assume X1<X2<X3<X4<X5, then accordingly, the electrical resistivities ρ1, ρ2, ρ3, ρ4, and ρ5 would have the relationship ρ1>ρ2>ρ3>ρ4>ρ5. See FIG. 1. Note that the curves shown in the figure are only for illustration purposes and the slope changes are exaggerated. It is noted that the slope of the alloy materials can vary depending on the degree of crystallinity. Specifically, in this embodiment, the slope becomes more positive as the degree of crystallinity decreases towards 0 (i.e., the degree of amorphicity increases). When the amorphicity becomes 0% (100% crystalline), the curve can become essentially a straight line with a positive slope. It is noted that at temperatures above the crystallization temperature Tx, a drastic drop in the electrical resistivity for each of the alloy samples is apparent. Also, because all of the samples become crystalline in this temperature regime, they can behave similarly, as evidenced in the collapse of all three curves in this embodiment.

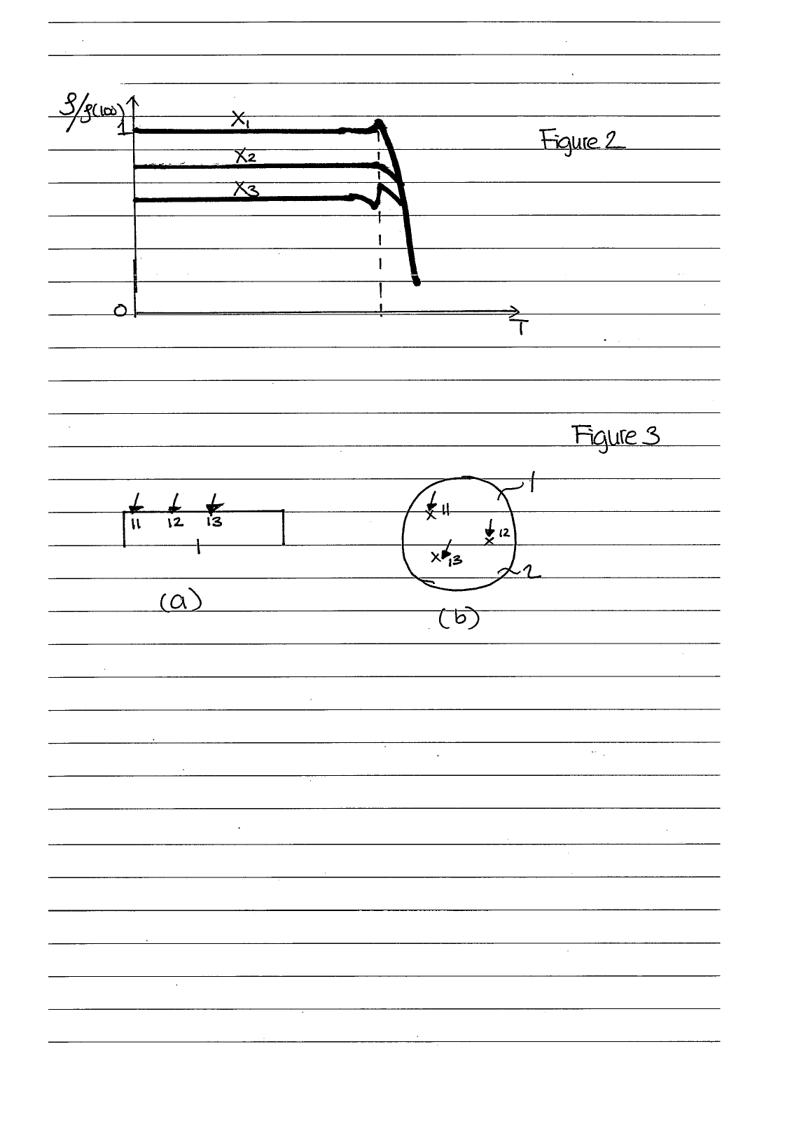

To facilitate comparison and illustration, in some embodiments, the data for each reference curve can be normalized by the data of a reference sample that is fully/completely amorphous (“ρ(100)”), as shown in FIG. 2. It is noted that other types of normalization can be used. For example, to account for the geometry of the sample, the electrical resistivity of each reference curve can be normalized by the dimension to examine the electrical resistance (as opposed to resistivity) of each reference sample.

Because of the temperature dependence, the electrical resistivity of the reference samples can be measured as the samples are heated. As a result, in addition to the electrical resistivity at a certain temperature, the entire heating history of the reference samples and/or test specimen can be compared. The heating can be carried by out by resistive heating, inductive heating, conductive heating, and/or radiative heating. In one embodiment, the heating can be conducted with an oven, heating pad, and/or a heating lamp. A radiative heating technique can be, for example, radiant heating, such as high intensity radiant heating. In one embodiment, the radiant heating can be carried out with an infrared lamp, such as a high density infrared lamp. The lamp can also be a plasma arc lamp, a tungsten-halogen lamp, or a combination thereof.

The electrical resistivity of each of the reference samples can be measured by any suitable technique. The technique can be destructive or non-destructive. In some embodiments herein, a “destructive” technique can refer to one that, by the performance of the measurement itself, permanently alters the sample in some way. For example, a BMG part being ground into powder and/or sectioned to be examined by an SEM. One consequence of a destructive measurement technique can be that the permanent alteration of the sample renders the sample unsuitable for a subsequent measurement or a subsequent measurement may give misleading results that are not representative of the sample. For example, a destructive technique can include differential scanning calorimetry (“DSC”). The techniques that can be used can include mechanical property measurement, radiography, microscopy, and differential scanning calorimetry (“DSC”). The methods can be destructive or non-destructive. A mechanical measurement can be carried out by bending, fracture toughness measurement, tensile test measurement, compressive test measurement, specific density measurement, hardness measurement. A radiography can be either x-ray radiography or x-ray diffraction. Etching can also be used. Microscopy can be any one of light microscopy, such as polarized light microscopy, and electron microscopy.

By contrast, a non-destructive technique does not alter the sample at least in a macroscopic sense, resulting in any observable change in the property of the sample. For example, a non-destructive technique would be to observe the sample visually without touching or touching the sample with a probe that creates no observable change in the sample. For example, a non-destructive technique can be to use a multi-meter to measure the resistance of a sample. In one embodiment where electrical resistivity of a reference sample is measured, a non-destructive technique can include a four-probe technique as described in Guo et al., Solid State Communications 135 (2005) 103-107. The technique can be also carried by a simple multimeter. An indirect resistance measurement can also be employed. In one embodiment, an inductive technique can be used. Some examples of an inductive technique can be one of those described in Teodorescu et al., International Journal of Thermophysics, vol. 22, No. 5, p. 1521 (2001) and Bakhtiyarov et al., Transactions of the ASME, vol. 126, p. 468 (2004).

The measurements can be carried out by suitable design of apparatus. For example, such an apparatus can comprise at least one of: multimeter, sample holder, electrodes; heater; temperature controller; electrical resistance measuring system; computer; and a display. The apparatus, or any of the aforementioned components thereof, can be portable.

Determining Crystallinity with a Master Curve Plot

The master curve plot with the plurality of reference curves can be used to examine a property of a specimen. For example, in one embodiment, using the relationship between the degree of crystallinity and the electrical resistivity for the plurality of the reference samples as a guide, the aforedescribed master curve plot can be subsequently used to determine the degree of crystallinity of an unknown alloy specimen. The alloy specimen and the reference alloy samples can be of any of the alloys described above.

As an exemplary embodiment, the master curve chart is used to determine the degree of crystallinity for an alloy specimen having the same chemical composition as the reference samples but an unknown degree of crystallinity. In this embodiment, the relationship between the electrical resistivity and the temperature of the alloy specimen can be measured by any of the aforedescribed techniques. Preferably, the technique used to determine the electrical resistivity of the alloy specimen is non-destructive. The relationship can be represented by a curve: The curve can then be superimposed onto the master curve plot. In some embodiments, because the electrical resistivity remains fairly independent of temperature but decreases with increasing crystallinity, the degree of crystallinity of the alloy specimen can be determined by comparing the location of the curve of the alloy specimen with those of the reference curves on a master curve plot.

In one embodiment, because of the temperature independence before crystallization temperature, Tx, the determination of crystallinity can be carried out at any pre-defined temperature below Tx. It is noted, however, that in some embodiments, it is preferred that the temperature is much lower than Tx. For example, depending on the alloy system, the degree of crystallinity can be determined at room temperature (about 300 K), 400 K, 500 K, 600 K, etc. It is noted that because of this temperature independence, one alternative embodiment of the presently described methods is to measure the resistivity of the alloy specimen at a single, pre-defined temperature below Tx, and compare that value with the master curve plot. In such an embodiment, the measurement can be fast because there would be no need to observe the entire heating history of the specimen. However, it is noted that obtaining the heating history of the specimen can provide additional valuable information about the alloy specimen as well.

Because of this independence, the temperature range within the master curve plot that can be used for the determination described herein can be fairly wide. For example, the temperature can be any temperature from 77 K, to crystallization temperature, such as from about 0° C. (273K) to crystallization temperature, such as room temperature up to crystallization temperature. The lowest temperature in the temperance can be lower than 0° C. as well, with no limit on the lowest temperature. For example, the lowest temperature in the range can be lower than about 250 K, such as lower than about 200 K, such as lower than about 150 K, such as lower than about 100 K, such as lower than about 80 K, such as lower than about 77 K, such as less than about 50 K, such as less than about 25 K, such as less than about 10 K, such as approaching 0 K.

Depending on the application, a temperature range far above the crystallization temperature can be used to allow the observation of thermal history of the alloy from below the crystallization temperature to above. For example, with the aforementioned lowest temperatures, the highest temperature in the temperature range can be at least about 100 K, such as at least about 150 K, such as at least about 200 K, such as at least about 250 K, such as at least about 300 K, such as at least about 350 K, such as at least about 350 K, such as at least about 400 K, such as at least about 450 K, above crystallization temperature. The temperature dependence can be obtained as a result of heating (e.g., from room temperature to crystallization temperature); or it can also be obtained as a result of cooling (e.g., from room temperature to 273 K or even lower to, for example, 77 K).

Additionally, because of the electrical resistivity dependence on crystallinity, the crystallinity of the alloy specimen of an unknown degree of crystallinity can be determined by comparing the curve with the reference curves. For example, if the curve of the alloy specimen falls between a reference curve of a reference sample that is 50 vol % crystalline and another that is 75 vol %, it can be postulated that the alloy specimen can have a degree of crystallinity that is between 50 vol % and 75 vol %. This range can be narrowed, the method further refined, by having more reference curves of different predetermined degrees of crystallinity.

Additional, more sophisticated methods can be employed to further refine the determination. For example, depending on the alloy system, the relationship between electrical resistivity and crystallinity can be elucidated by a certain mathematical formula (e.g., linear, quadratic, exponential, polynomial, etc. function). Accordingly, in one embodiment, by determining the relative position of the curve of the alloy specimen with respect to the reference curves, the precise degree of crystallinity of the alloy specimen can be determined based on the mathematical formula. In another embodiment, the comparison can be carried out for the positions of the curves at a specific pre-defined temperature, instead of an entire curve. Alternatively, the comparison can also be carried out for a portion of the curves, instead of the entire curves or a single point.

Because a master curve plot is chemical composition specific, more than one master curve plot can be used simultaneously in the case of an alloy specimen of a different chemical composition to be examined. For example, a second master curve plot for a series of reference alloy samples of a second composition, a third plot, a fourth plot, and the like can be superimposed and used simultaneously. Alternatively, the different master plots can be used separately.

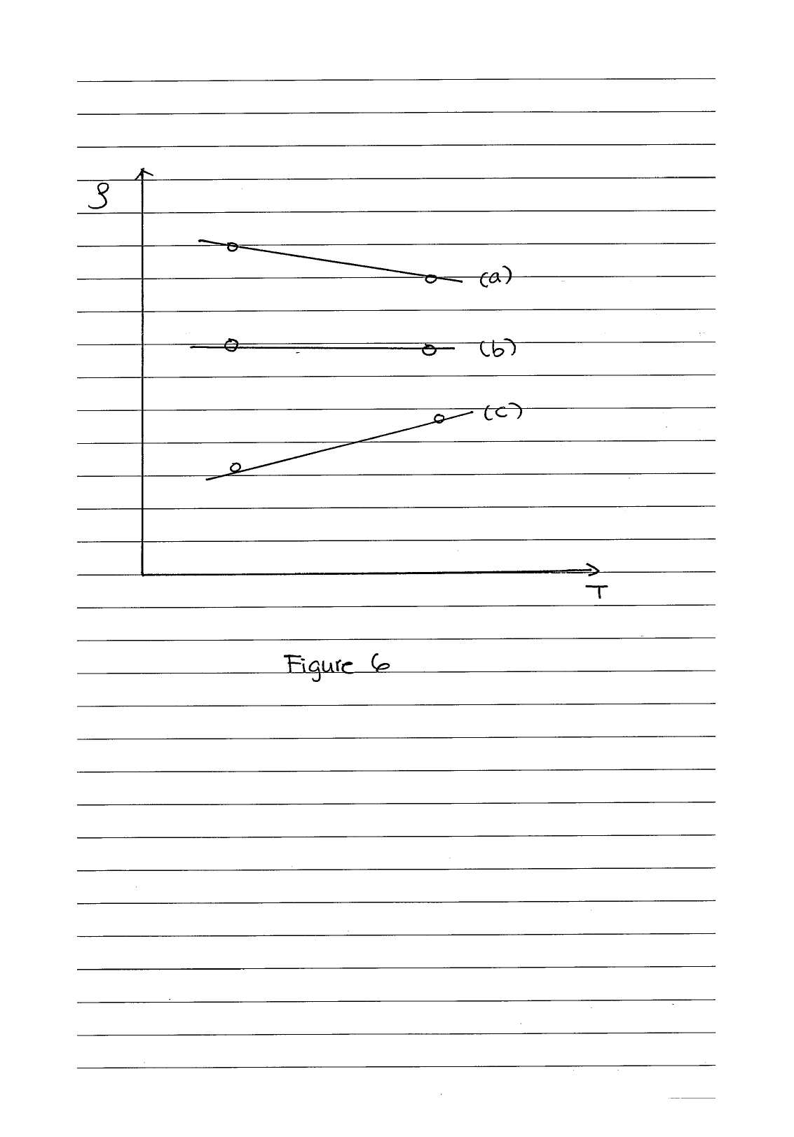

The presently described methods can also be used to evaluate an alloy specimen without the use of a master curve. For example, in one embodiment, because it is known that a crystalline alloy tends to have a positive slope for its resistivity vs. temperature curve, the slope of a ρ vs. T curve of the alloy can provide an indicator of the property of the alloy. In one embodiment, an alloy can be measured for its resistivity at at least two temperatures (e.g., at least 3, at least 4, etc). By plotting the measurement data on an ρ vs. T plot, these data points can be connected by a line, as shown in FIG. 6. In one embodiment, because crystalline alloys are known to show a positive slope (e.g., FIG. 6(c)), any alloy that has a line showing a zero slop or a negative slope (e.g., FIG. 6(b), 6(a), respective), can be deemed as non-crystalline (or at least partially non-crystalline). In this embodiment, no master curve is needed. Alternatively, a master curve can also be used. In another embodiment, the correlation between a certain degree of crystallinity and slope can be predetermined. Accordingly, by measuring the slope of the curve and comparing that with a predetermined value, the degree of crystallinity of the alloy specimen can be determined based on the slope. Also, the material property provided by this method need not be a precise degree of crystallinity. For example, a positive slope can indicate the presence of crystals, thereby providing some indications of the alloy specimen.

### Quality Control

Depending on the application, a BMG having any crystallinity can be undesirable. On the other hand, it is equally undesirable to destroy the BMG part in order to measure its crystallinity. Thus, it is desirable to monitor and/or measure the degree of crystallinity of an amorphous alloy sample non-destructively. The aforedescribed methods involving using a master curve plot can be useful. FIG. 4 provides an illustrative flow diagram showing the steps of using the master curve plot to determine the crystallinity of an alloy specimen in one embodiment.

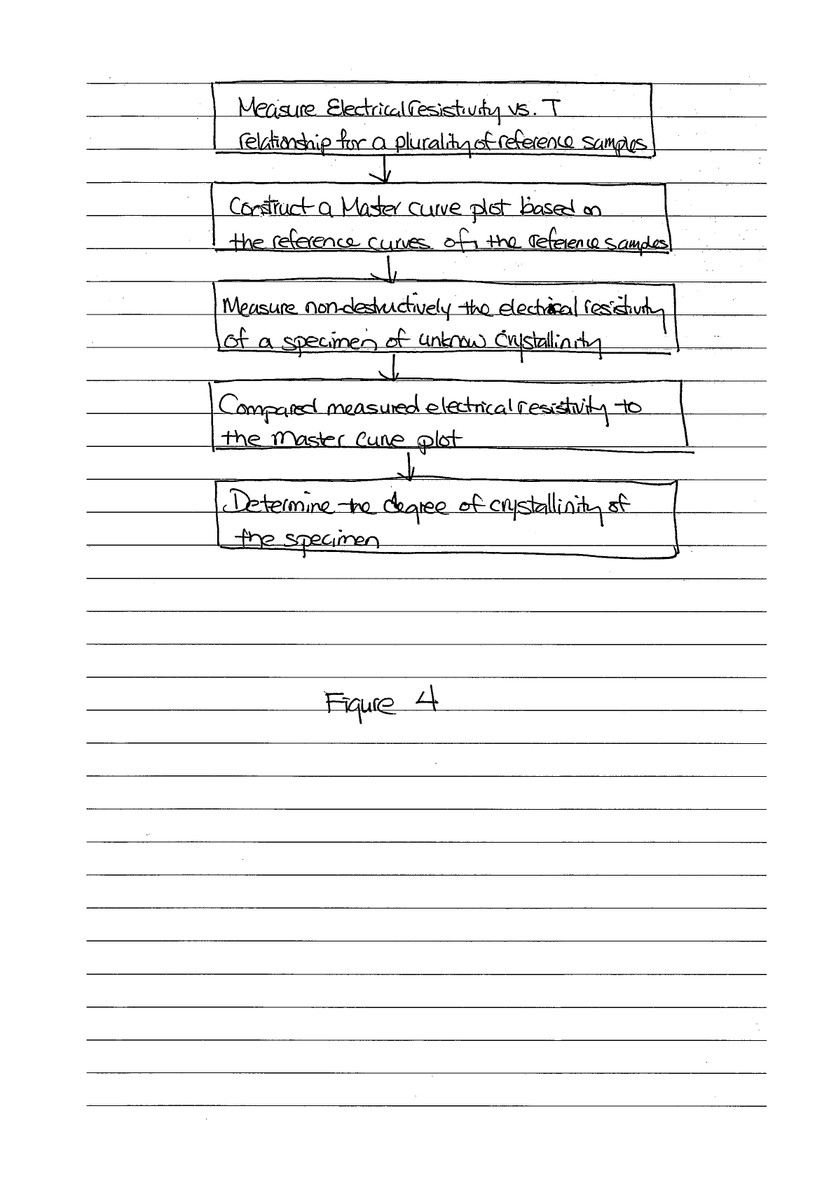

The electrical resistivity can be measured at different locations on an alloy specimen. The alloy can be any of the alloys described above, such as fully amorphous, or partially amorphous. Such series of measurements can be useful to ensure uniformity of crystallinity. For instance, crystals may occur randomly and uniformly in the alloy specimen. In one embodiment, a series Of electrical resistivity measurements can be carried along a certain axis on any surface of an alloy sample at multiple locations 11, 12, 13 of BMG 1, as shown in FIG. 3(a). Alternatively, these locations can be on a plane 2 (or cross-sectional area) of BMG 1, as shown in FIG. 3(b).

Along the same line, the presently described methods can further be used to correlate a material property with degree of crystallinity. The present methods can be particularly useful to provide an indicator of “local” property of the material, as opposed to a global measurement of the alloy specimen as a whole. For example, locations 11, 12, and 13 in FIGS. 3(a)-3(b), as opposed to the entire BMG 1 as a whole. For example, the presently described methods will be able to provide a crystallinity measurement of the top side, left side, right side, bottom side, etc. of an alloy specimen, as opposed to a pre-existing (destructive) method that can only provide measurement of a crystallinity measurement of the alloy specimen as a whole after a DSC measurement.

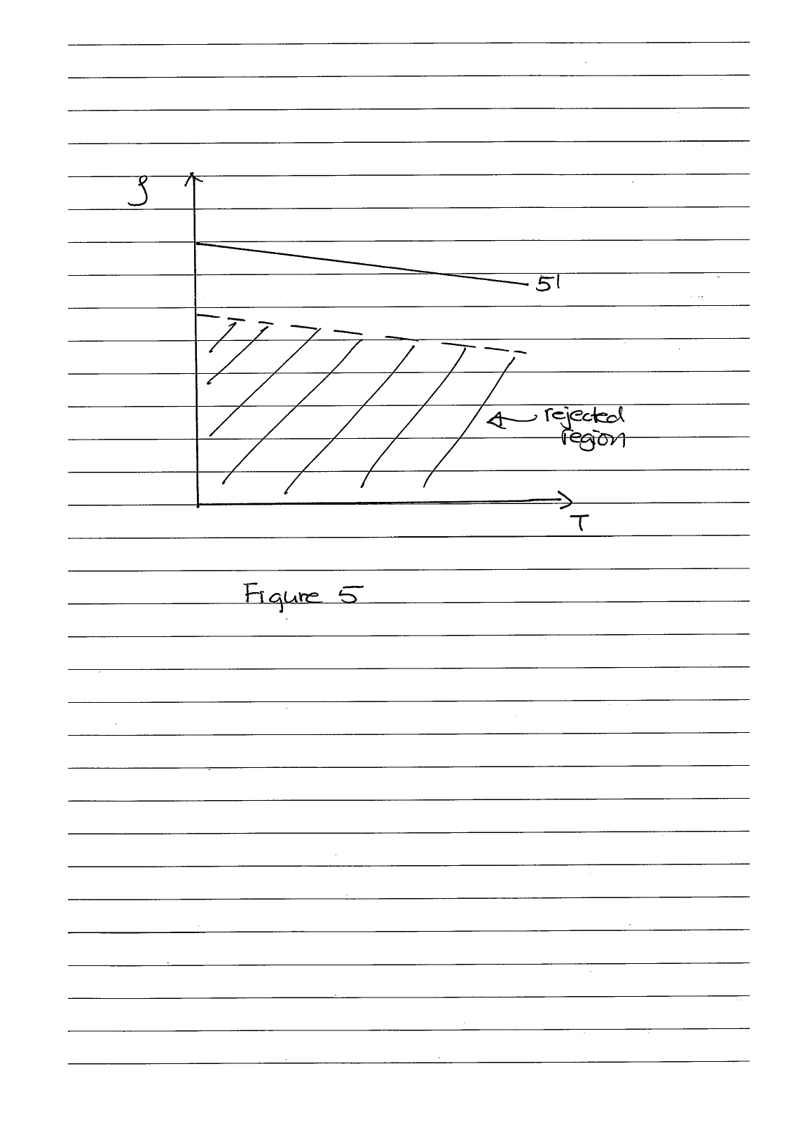

The presently described methods can provide a non-destructive way of monitoring and/or measuring the quality of an alloy product particularly in an alloy fabrication process in terms of the degree of crystallinity. For example, an additional monitoring step can be added. In one embodiment, criteria of qualities to be considered as “good” and “bad” can be introduced such that a binary determination of “good” and “bad” product can be added after the determination of the degree of crystallinity is further conducted. Such binary indicators can be used to accept or reject a product. This quality measurement/determination can also be used to evaluate the process of making the alloy, as shown below, thereby allowing modification and/or optimization of the making process. For example, the temperature dependence of the electrical resistivity of an alloy sample can be used to evaluate the quality or fabrication (i.e., making) process of the alloy. For example, as shown in FIG. 5, if curve 51 represents the relationship of an alloy that is deemed “acceptable” or “good,” then an alloy with a relationship that is this level, such as that shown in the shaded region, can be deemed as “bad” or “not acceptable.” The bad or not acceptable alloys can be rejected. The level of good or acceptable can be arbitrarily defined, so can the shaded region.

Similarly, in the embodiment described above, wherein on master curve is used, the alloy with a non-positive slope for its ρ vs. T curve can be deemed “acceptable,” while the former can be deemed “not acceptable.” Accordingly, without the need to use a master curve, the slope of the line can be used to determine a material parameter (e.g., crystalline or not crystalline) of the alloy, or even to evaluate the efficacy of the fabrication process of making an amorphous alloy, as aforedescribed.

An apparatus that can carry out the aforedescribed methods can be employed as a quality control apparatus, which can be integrated with the metallic glass fabrication system. For example, if the indicator in the apparatus shows a “bad” signal, the apparatus can send a signal to the fabrication system to stop the process. Alternatively, the signal can be sent to the fabrication system to modify the fabrication conditions (e.g., temperature, pressure, etc.) to optimize the quality of the product. The quality control feedback of the system integrated with the apparatus can be continuous—i.e., the feedback is continuously sent and received by the fabrication system and adjustments are made continuously. Alternatively, the feedback can be discrete—i.e., the signal is sent at a specified time and the fabrication system can be monitored and examined then based on the feedback.

Furthermore, instead of a binary “good” or “bad” determination, the presently described methods can be used to describe directly the levels/degrees of crystallinity. In one embodiment, such post-processing/fabricating determination can provide feedback in the fabrication process such that the fabrication parameters can be adjusted to optimize the product quality. In one embodiment, the entire process that includes fabrication, quality monitoring, and parameter adjustment can be automated, i.e., controlled by computer.

The aforedescribed quality control can be valuable in the fabrication process involving using BMG. Because of the superior properties of BMG, BMG can be made into structural components in a variety of devices and parts. One such type of device is an electronic device.

An electronic device herein can refer to any electronic device known in the art. For example, it can be a telephone, such as a cell phone, and a land-line phone, or any communication device, such as a smart phone, including, for example an iPhone™, and an electronic email sending/receiving device. It can be a part of a display, such as a digital display, a TV monitor, an electronic-book reader, a portable web-browser (e.g., iPad™), and a computer monitor. It can also be an entertainment device, including a portable DVD player, conventional DVD player, Blue-Ray disk player, video game console, music player, such as a portable music player (e.g., iPod™), etc. It can also be a part of a device that provides control, such as controlling the streaming of images, videos, sounds (e.g., Apple TV™), or it can be a remote control for an electronic device. It can be a part of a computer or its accessories, such as the hard drive tower housing or casing, laptop housing, laptop keyboard, laptop track pad, desktop keyboard, mouse, and speaker. The article can also be applied to a device such as a watch or a clock.

The articles “a” and “an” are used herein to refer to one or to more than one (i.e., to at least one) of the grammatical object of the article. By way of example, “a polymer resin” means one polymer resin or more than one polymer resin. Any ranges cited herein are inclusive. The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

What is claimed:

1. A method of determining an unknown degree of crystallinity, comprising:
constructing a master curve plot comprising a plurality of reference curves, each reference curve representing a relationship between electrical resistivity and temperature for one of a plurality of reference alloy samples having a chemical composition and various predetermined degrees of crystallinity;
for an alloy specimen having the chemical composition and the unknown degree of crystallinity, obtaining a curve representing a relationship between electrical resistivity and temperature thereof; and
determining the unknown degree of crystallinity by comparing the curve to the master curve plot.

2. The method of claim 1, wherein the alloy comprises Zr, Hf, Ti, Cu, Ni, Pt, Pd, Fe, Mg, Au, La, Ag, Al, Mo, Nb, Be, or combinations thereof.

3. The method of claim 1, wherein the step of obtaining the curve for the alloy specimen is carried out by a non-destructive technique.

4. The method of claim 1, wherein the step of constructing further comprises measuring the relationship for each of the plurality by a destructive technique.

5. The method of claim 1, wherein the step of constructing further comprising normalizing each reference curve with respect to the reference curve representing a fully amorphous reference alloy sample.

6. The method of claim 1, wherein the temperature ranges from 77 K to at least about 100 K above a crystallization temperature of the alloy.

7. The method of claim 1, wherein the step of determining further comprises superimposing the curve for the alloy specimen onto the master curve plot.

8. The method of claim 1, wherein the step of determining further comprises comparing the curve for the alloy specimen to the master curve plot at a predefined temperature.

9. The method of claim 1, further comprising constructing a second master curve plot for a plurality of reference alloy samples having a different chemical composition.

10. The method of claim 1, wherein the method is carried out in an apparatus comprising at least one of: multimeter, sample holder, electrodes; heater; temperature controller; electrical resistance measuring system; computer; and a display.

11. A method of determining an unknown degree of crystallinity, comprising:
providing a plurality of reference alloy samples of a chemical composition, each of the plurality having a predetermined degree of crystallinity;
for each of the plurality, measuring a relationship between electrical resistivity and temperature to obtain a reference curve representing the relationship;
constructing a master curve plot comprising at least some of the reference curves;
providing an alloy specimen having the chemical composition and the unknown degree of crystallinity;
for the specimen, measuring a relationship between electrical resistivity and temperature to obtain a curve representing the relationship; and
determining the unknown degree of crystallinity for the specimen by comparing the curve to the master curve plot.

12. The method of claim 11, wherein the step of measuring for each of the plurality further comprises measuring the electrical resistivity while heating the reference alloy sample.

13. The method of claim 11, wherein the step of measuring for each of the plurality further comprises heating the reference sample by resistive heating, inductive heating, conductive heating, radiative heating.

14. The method of claim 11, wherein the step of measuring for each of the plurality is carried out by at least one of bending, x-ray radiography, x-ray diffraction, etching, light microscopy, electron microscopy, specific density measurement, hardness measurement, fracture toughness measurement, tensile test measurement, and differential scanning calorimetry.

15. The method of claim 11, wherein the step of measuring for the alloy specimen is carried out by measuring the electrical resistivity thereof by a four-probe technique, an inductive technique, or a combination thereof.

16. The method of claim 11, wherein the step of determining comprises comparing the curve to the master curve plot at a pre-defined temperature.

17. The method of claim 11, wherein the step of measuring for the specimen is repeated at different locations on the specimen.

18. The method of claim 11, further comprising measuring the unknown degree of crystallinity for the specimen at a plurality of locations on the specimen.

19. The method of claim 11, further comprising measuring at least one material property and correlating the property to the determined unknown degree of crystallinity.

20. The method of claim 11, further comprising making the alloy specimen and evaluating the step of making based on the comparison of the curve and the master curve.

21. A method of evaluating an alloy specimen, comprising:
measuring the electrical resistivity of the alloy specimen at at least two temperatures;
determining a slope of a line connecting the electrical resistivity measurements on a resistivity versus temperature plot; and
evaluating the alloy specimen based on the slope.

22. The method of claim 21, further comprising determining a degree of crystallinity of the alloy specimen based on the slope.

23. The method of claim 21, further comprising making the alloy specimen and evaluating the making of the alloy based on the slope.
